(19) United States
(12) Patent Application Publication
(10) Pub. No.: US 2002/0032421 A1
(43) Pub. Date: Mar. 14, 2002

(54) NONWOVEN, ABSORBENT FIBROUS WEB AND METHOD OF MANUFACTURE

(76) Inventors: JOHN B SCOTT JR., GERMANTOWN, TN (US); MAURICE R BAILEY JR., BARTLETT, TN (US)

Correspondence Address: DARBY & DARBY, 805 THIRD AVENUE, NEW YORK, NY, 10022

(21) Appl. No.: 08/997,599

(22) Filed: Dec. 23, 1997

Related U.S. Application Data
(60) Provisional application No. 60/044,794, filed on Apr. 24, 1997.

(51) Int. Cl. A61F 13/15; A61F 13/20

(52) U.S. Cl. 604/367; 604/365; 604/366; 604/368; 604/370; 604/372; 604/374; 604/375

(57) ABSTRACT

Disclosed is a nonwoven, absorbent fibrous web, including a layer of airlaid substantially opened fibers. The fibers include more than 70% by weight short fibers. Also included in the web is an interfiber binder.

Also disclosed is a method for making a nonwoven, absorbent fibrous web. The method includes the steps of (1) providing a source of substantially opened fibers, said fibers including more than 70% by weight of short fibers; (2) introducing an interfiber binder to said sources of fibers; (3) airlaying said source of fibers to form a layer of fibers; and (4) activating said binder to cause formation of interfiber bonds.

This application claims priority under 35 U.S.C. §119 from U.S. Provisional Patent Application Serial No. 60/044,794, filed Apr. 24, 1997, the disclosure of which is incorporated herein by reference.

### FIELD OF THE INVENTION

The present invention relates to a nonwoven, absorbent fibrous web of short fibers. More particularly, the invention relates to an absorbent web of short cellulose fibers and a method for its manufacture, which webs are useful for the manufacture of feminine hygiene tampons, interlabial devices, and other pledgets, such as those used in dentistry and in bandages.

### BACKGROUND OF THE INVENTION

Feminine hygiene tampons are constructed from a fibrous web or band of long cotton fibers, long rayon fibers and mixtures thereof. The web may also contain a minor amount of short cotton fibers. The fibrous web is prepared by processing loose fibers through various arrangements of opening, carding and sometimes combing to form a continuous fibrous web.

For the manufacture of tampons and other pledgets (as used herein, unless otherwise indicated, “tampons” refers both to feminine hygiene tampons and other pledgets) intended for contact with mucous membranes or wound sites, all natural, cellulose-based fibers, namely rayon and long staple cotton are employed. Cotton fibers and rayon fibers are used because each are absorbent and have high purity (as measured by alpha cellulose content). Synthetic fibers (such as polyester and polypropylene) are not as absorbent and wood-based cellulose fibers are not as high in purity. High purity fibers are less likely to provide a medium for bacterial growth.

Conventionally, the fibrous web is formed by textile processing equipment, and is further treated by needlepunching and/or calendering to increase the tensile strength of the web to a level sufficient to enable the web to be subsequently successfully converted by conventional tampon forming machines.

Fiber is typically transported in large dense bales. Thus, the first step in preparing the web requires “opening” to individualize the fibers. The density of the material must be uniformly reduced to provide a suitable feedstock for the subsequent mat formation step. Opening equipment disintegrates the high density material from the bales to form a light fluffy material which may be conveyed in an air stream and consolidated into a low density feedstock for the mat formation step.

The raw fibers formed into a low density feedstock are then carded to align the fibers and remove waste material. The card includes rotating cylinders each with protruding fine wires or hooks. The card is used to draw apart the fibers and create a fiber mat of substantially parallel fibers.

After carding and/or combing, the web is wide, thin and has little strength. Typically, to form a tampon band, the web is collapsed by folding it inwardly upon itself to form a band of suitable width for conversion on tampon production machinery. The collapsed web is too thick (generally 5-10 mm) and is not strong enough for tampon converting operations. Embossing and calendering are each steps which can reduce the thickness of the web and increase its strength. Calendering reduces the web thickness by pressure applied by passing the web through opposed, loaded smooth rolls. Embossing is the same as calendering but the rolls have a raised pattern which imparts localized pressure and hence localized, high density on the web. Optionally, the web may be needlepunched before the calendering or embossing step. Needlepunching entails entangling the long fibers of the web by punching the web in the Z direction with barbed needles.

The opening, carding and calendering steps using conventional textile processing equipment to form webs suitable for tampon conversion requires that the fiber content include at least about 30% by weight of long fibers.

A conventional airlaid web is typically prepared by disintegrating or defiberizing a pulp sheet or sheets typically by hammermill to provide substantially opened fibers. The opened fibers are then air conveyed to forming heads on the airlaid web forming machine. Several manufacturers make defiberized pulp sheet airlaid web forming machines, including M&J Fibretech of Denmark and Dan-Web, also of Denmark. The forming heads include rotating or agitated drums, generally in a “race track” configuration which serve to maintain fiber separation until the fibers are pulled by vacuum onto a foraminous condensing drum or foraminous forming conveyor. Other fibers, such as a synthetic thermoplastic fiber, or superabsorbent fiber may also be introduced to the forming head through a fiber “dosing” system which includes a fiber opener, a dosing unit and an air conveyor. Non-fibrous materials, such as super-absorbent polymer (SAP) granules, may also be added to the forming head by a dosing system.

The airlaid web is transferred from the condensing drum or forming conveyor to a calender or other densification stage to densify the web, increase its strength and control web thickness. The fibers of the web are then bonded by application of a latex spray or foam addition system, followed by drying or curing. Alternatively, or additionally, any thermoplastic fiber present in the web may be softened or partially melted by application of heat to bond the fibers of the web. The bonded web may then be calendered a second time to increase strength or emboss the web with a design or pattern. If thermoplastic fibers are present, hot calendering may be employed to impart patterned bonding to the web. Water may be added to the web if necessary to maintain specified or desired moisture content, to minimize dusting and to reduce the buildup of static electricity.

The tampon bandstock (or feedstock) prepared by carding is converted to finished tampons by a tampon forming machine such as that manufactured by Karl Ruggli AG of Fisibach, Switzerland. Other such machines are made by Hauni Richmond of Richmond, Va. or are manufactured to the specifications of the tampon producer. In the machine, the bandstock is separated to bands of a predetermined length. In a preferred embodiment, a nonwoven cover sheet is placed on one side of the tampon band, and a cotton withdrawal string is attached to the band. The band is then wound into a cylinder having substantially concentric layers, and having the non-woven cover sheet on the exterior of the cylinder. The cylinder is first compressed radially, and then by longitudinal compression, while circumferential compression is maintained. Following these compression steps, the finished tampon is then packaged, optionally with an application tube.

Alternatively, the tampon may be prepared by lateral compression. Bandstock is cut to size, a nonwoven sheath is wrapped around the cut core, the removal string is stitched in place, and the unit is laterally compressed, followed by longitudinal compression prior to insertion into an application tube.

Tampons have conventionally been manufactured from long cotton or rayon fibers, as opposed to short fibers such as cotton linter fibers, short cotton staple fiber, or other short fibers, such as ground or fractioned cotton staple, rayon or high-alpha cellulose wood pulp. Long cotton fibers typically have a length of about 12-45 mm. As used herein “short fibers” are defined as having a length of about 0.5-12 mm. Short fibers include vegetable fibers, e.g. cotton, regenerated cellulose (rayon or Lyocell), synthetic long chain polymers, cotton linters, cotton noils, gin motes, and long cotton, rayon or other fibers which have been reduced in length to 12 mm or less. Both long cotton fibers and cotton short fibers have a diameter of 0.02-0.04 mm.

Although equivalent to long cotton fibers in composition and diameter, cotton short fibers are less expensive because their short fiber length limits their suitability for many commercial applications. Prior to the present invention, cotton short fibers could not be employed in amounts of more than 70% by weight, in tampon production because of processing difficulties in conventional textile equipment and difficulties in providing sufficient tensile strength in the resultant bandstock. It was previously thought that cotton short fibers were too short to maintain the required structural integrity of both the band-stock during tampon production, and of the final tampon product. This is because structural integrity in carded bands and the tampons made from such bands is generally imparted in large part by the frictional forces between fibers. Longer fibers interact with a greater number of other fibers than short fibers, and so, when used alone, provide a structure having a higher tensile strength than is possible with shorter fibers. For this reason, until the present invention, it has not been possible to use less than about 30% by weight of long cotton, rayon, or other staple fibers.

Interaction of fibers can be fostered by carding, calendering, embossing and needlepunching processes, each of which orients the fibers in the machine direction (or in the case of needlepunching entangles the fibers in the Z direction). This orientation provides tensile strength for processing through the tampon machine.

Another reason that short fibers have not been used in amounts exceeding 70% by weight for tampon manufacture is that it was thought that a high proportion of short fibers would present an insurmountable and commercially unacceptable “dust-off” problem. Dust-off occurs when individual fibers are separated from the web or finished tampon.

Thus, although short fibers have been employed in tampon manufacture, they have not been used as the predominant fiber source, i.e., more than 70% by weight.

### SUMMARY OF THE INVENTION

The present invention is directed to a nonwoven, absorbent fibrous web, including an airlaid layer formed from substantially opened fibers. The fibers include more than 70% by weight short fibers. Also included in the web is an interfiber binder.

The present invention is also directed to a method for making a nonwoven, absorbent fibrous web. The method includes the steps of (1) providing a source of substantially opened fibers, said fibers including more than 70% by weight of short fibers; (2) introducing an interfiber binder to said source of fibers; (3) airlaying said source of fibers to form a web of fibers; and (4) subjecting said binder to conditions sufficient to cause formation of interfiber bonds.

### DETAILED DESCRIPTION OF THE INVENTION

Prior to the present invention, it was thought in the art that a commercially manufactured tampon could not be made from a web containing more than 70% by weight short fibers, such as cotton short fibers, due to the limitations imposed by conventional textile processing equipment. Likewise, it was not previously thought that a tampon product made from a web of predominantly short fibers would provide satisfactory performance characteristics.

It has now been surprisingly and unexpectedly discovered that, by using airlaid web forming technology, a web may be made from a fiber source of predominantly short fibers together with a binder. The resultant web, converted to bandstock, has sufficient tensile strength to be converted into tampons on conventional tampon forming machinery. Further, the tampons so produced have performance characteristics comparable to those of tampons made from long cotton or rayon fibers. For example, the tampon of the present invention has a high density absorbent structure that meets the FDA standards as set forth in the Syngyna Absorbancy Test, the fundamental standardized performance requirement for feminine hygiene tampons. Additionally, conventionally processed tampons have a tendency to lose fibers (shed) when wet due to the use of fiber to fiber friction as a binder system. In contrast, the tampons of the present invention do not shed fibers, as fiber to fiber friction is not the primary binder. Moreover, the tampons of the present invention permit better control of basis weight, tensile strength, and absorbency in comparison to conventionally processed tampons.

By using less expensive short fibers, such as cotton linters, rather than long cotton or rayon fibers, the fiber cost of the tampon may be substantially reduced, and there is a substantial increase in the economy of scale of the production of tampons, as well as an elimination of the need for conventional textile processing equipment at the converting site, as the bandstock of the present invention may be shipped to the converting site and used directly.

By employing an interfiber binder, such as thermoplastic fiber in the web, the short fibers may be bonded together. The tensile strength of the web may be adjusted as necessary by adjusting the proportion of short fibers to binder.

The airlaid web of the present invention is prepared by disintegrating or defiberizing a pulp sheet or sheets of cotton linters or other short fibers to produce opened short fibers. Although sheeted pulp sheets are preferred, bulk pulp can be substituted in whole or in part by processing through standard textile fiber opening and dosing systems and air conveying to the forming heads. The opened fibers are then air conveyed to forming heads on the airlaid web forming machine. An interfiber binder, such as a synthetic thermoplastic fiber is also introduced, preferably at the forming head, through a fiber “dosing” system. Preferably, the binder is a thermoplastic fiber. The preferred thermoplastic fiber is Celbond Type 255 Bico fiber from Hoechst Celanese. The Bico fiber has a polyester core (approximately 50% of content of fiber) and an activated co-polyolefin sheath (50% of fiber), wherein “activated” implies that the co-polyolefin sheath has been chemically modified to promote adhesion of dissimilar materials. The Bico fiber has a melting point of 128° C., displays a shrinkage of 3% at 110° C., and has a 3 denier. The Bico fibers have approximately a 5 mm cut length, and are low crimp fibers. Although 5 mm is a preferred thermoplastic fiber length, the fibers may be of any suitable length, generally between about 0.5 and 12 mm, including mixtures of fibers of differing lengths. The purpose of the thermoplastic fiber is to bind the short fibers together upon softening or partial melting of the thermoplastic fiber. After the short fibers are bound together by the softened or partially melted thermoplastic fibers, the web is cooled and the thermoplastic fibers harden, permanently binding the short fibers together.

Any thermoplastic fiber may be used provided that the softening or partial melting temperature is sufficiently low that the cotton or other short fibers are not damaged. Further, the thermoplastic fiber should solidify readily upon minimal cooling. Examples of other suitable thermoplastic fibers include polyolefins such as polyethylene and polypropylene, and polyesters, polyamides, nylons and acrylics.

Although the fiber form is preferred, the thermoplastic material may alternatively be provided in the form of granules, pellets, powder, flakes, chips or any other physical form which allows sufficient intimate contact with the short fibers to permit bonding of the short fibers upon application of heat to the fiber/thermoplastic mixture.

In addition to thermal fusion bonding, the fibers of the airlaid web may also be bonded by adhesives, solvents or physical entanglement. Suitable adhesives include cellulose acetate, polyvinyl acetate, starch-based binders, e.g., amphoteric, dialdehyde, cationic (dry strength) and anionic (wet strength) binders, carboxymethylcellulose, and latex emulsions. Latex emulsions may include styrene butadienes, ethyl vinyl acetates, acrylates and methacrylates, vinyl acetates and mixtures thereof. Alternative binders include poly(vinyl alcohols), polyamide epichlorhydrin resins and polyacrylamides. Solvent binders include cellulose acetate solutions and cellulose acetate treated with an activator, e.g., Triacetin, (CH₃CO₂CH₂)₂CH(O₂CCH₃) (CAS 102-76-1), available from Aldrich Chemical Company, St. Louis, Mo. Approximately 10 to 40% (of the fiber weight) binder adhesives may be employed, and binders may be activated by various methods, including water evaporation, crosslinking, and thermal setting. Additionally, water soluble binders may be used for flushability.

If desired, additional absorbent materials, such as super-absorbent polymer (SAP) granules, fibers, flakes, or foams may be included in the web by addition to the --forming head by a suitable dosing system. Super-absorbent polymer refers to one or more hydrocolloid materials capable of absorbing many times their own weight of aqueous fluid. These materials are generally water soluble or water swellable polymers, prepared by polymerization of suitable monomers, leading to the formation of homopolymers, or copolymers. The polymers are typically reacted with a crosslinking agents during and/or after polymerization to form crosslinked polymers, which confers a degree of water insolubility to otherwise water soluble polymers, while retaining susceptibility of the polymer to swelling in water and water-containing fluids. Typically, the superabsorbent polymers are salts of poly (acrylic acid) or acrylic acid copolymers. Additional superabsorbents include hydrophilic polymer grafts onto starch or cellulose backbones, crosslinked carboxylated celluloses, and the salts of maleic anhydride copolymers.

Other materials which may be added include perfumes, colorants, surfactants, antimicrobial agents, odor control agents (e.g., sodium bicarbonate, silica gel), zeolites and the like. If the additive is sensitive to subsequent bonding treatment (such as the application of heat to the web for thermoplastic fibers) the additive may be applied to the web downstream of such treatment.

The fibers of the airlaid web may alternatively be bound by hydroentanglement (or spunlacing) by directing a very fine, high pressure water jet towards the airlaid web to cause an physical entanglement of the fibers, supported by, for example, a 60×40 mesh support wire. Current hydroentanglement processes enable one to achieve a maximum waterjet pressure of 300 bar (4400 psi), a basis weight range of 20-400 gsm, maximum production speed of 300 m/min, a bonding energy of 0.30 KWH/kg, 2% fiber loss and excellent reliability.

As discussed above, short fibers constitute more than 70% by weight of the fibers in the web of the present invention. The short fibers may be provided as one type or as a mixture of two or more different fiber types. The balance of the fibers may be cotton or rayon staple fibers or any other long fiber, e.g., fibers longer than approximately 12 mm in length, including cellulose regenerated from amine oxide solution (NMMO), such as Lyocell.

In a preferred embodiment, cotton short fibers and thermoplastic fibers are laid onto the forming conveyor or condensing drum of an airlaid machine by vacuum in a homogenous mixture having a proportion by weight of between about 80/20 to 99/1 of cotton short fibers to thermoplastic fibers. Preferably the ratio is about 93/7 cotton short fibers to thermoplastic fibers. However, the ratio may be adjusted as necessary to provide a desired tensile strength.

In a second preferred embodiment, the fibers in the nonwoven, absorbent fibrous web are substantially opened. The method of the present invention provides substantially opened fibers which are loosely oriented, rather than hard and paper like. Fibers are opened by either disentanglement, i.e. separating fibers into their individual elements, or separation of compressed and matted masses into loose tufts. Preferably, the fibers are opened by the latter method. The fibers should be substantially opened such that a mat is formed having sufficient integrity on the forming wire of the airlay machine to allow subsequent transfer to the oven to melt the outer sheath of the Bico fiber.

In a third preferred embodiment, an airlaid web of homogeneous cotton short fibers is prepared without the addition of thermoplastic or any other fibers. In other words, short fibers account for 100% by weight of the fibers present in the web. The short fibers of the web can be bonded by application of latex foam or spray, followed by drying and curing.

In a further preferred embodiment, an airlaid web comprising a mixture of cotton short fibers and cotton or rayon long fibers is hydroentangled using high pressure water jets, such that absorbent fibers account for up to 100% by weight of the web. The short fibers are bonded by physical entanglement with the longer fibers and other short fibers. Hydroentanglement is followed by drying.

The airlaid web may be transferred to a calender to increase its density and strength and adjust web thickness, to a predetermined thickness, preferably to 3 mm. The bonded web may subsequently be hot calendered to further increase strength or to emboss the web with a design or pattern. Water may be added to the web if necessary to maintain specified moisture content. The web is then rolled and slit to a desired width suitable to form bandstock for use in tampon forming machinery.

The following Examples are provided to illustrate the invention, and are not to be construed in any way as limitations thereon. All obvious modifications, changes, and adaptations of the invention are intended to be within the meaning and range of equivalents of the appended claims.

### EXAMPLE 1

### Production of Airlaid Band From Cotton Linters and Thermoplastic Fiber

Several forms of current commercial tampon bands were compared to a sample of an embodiment of the airlaid tampon band of the invention.

The airlaid tampon band of the invention was produced on an M&J airlaying line by disintegrating by hammermill cotton linter cellulose sheet, Grade 1NR75 (obtained from Buckeye Cellulose Corporation, Memphis, Tenn. ), and air conveying the resultant fibers to forming heads. Hoechst Celanese's Celbond Type 255 Bico fiber (a bicomponent fiber with a polyester core and a activated copolyolefin sheath) was dosed into an air stream and conveyed to the forming heads and blended with the cotton linter cellulose. The amount of the Bico fiber was 15% of the total weight of the airlaid web. The resultant airlaid web was lightly calendered to reduce thickness to 3 mm, increase density and slightly increase tensile strength. The web was then passed through thermal bonding ovens. The temperature of the ovens was set to a temperature above the melting point of the Bico fiber, with sufficient dwell time in the ovens to allow completion of bonding to produce a thermally bonded airlaid web, e.g. 130-190° C and a dwell time of about 30 seconds. The web was then wound and slit to a width of 50 mm.

Buckeye Cotton Linter Cellulose sheet (1NR75) has the following typical properties ISO brightness—88%; moisture content—6.5%; ream weight—350 lbs.; sheet thickness—0.050 inches; Clark fiber length on 14 mesh—43%; sheet Mullen—50 psig; and weighted average fiber length—1.91 mm.

### EXAMPLE 2

Bands of various composition were compared for their physical and absorbent properties. Liquid absorbency is largely a factor of density, and improves with decreasing density. As seen in Table 1 below, the density of the band of Example 1 is comparable to the lowest density commercial band analyzed. Tensile strength of the band is important to converting reliability. If the tensile strength is too low, forces on the web during converting operations can result in a break in the web, and resultant machine downtime. Similarly, if the tensile strength is too high, the band will not break where required. If the band does not break where and when intended, the result will be an overweight tampon, which will cause a machine jam. As seen in Table 1 below, the band of Example 1 has a tensile strength comparable to commercially available bands. The grams per linear meter is a measurement of the basis weight per unit area of the tampon band. The airlaid process of the present invention provides a more uniform and easily controlled weight per unit length. As a result, the final tampon product will have a more uniform and specified weight. Tensile strength and percent elongation were measured by standard testing on a Thwing Albert Intelect II Tensile Tester in units of g/in. Converters and tampon machine manufacturers (Ruggli AG in particular) have processed bands of 105 g/in. There is no known maximum except that set by tampon manufacturers with known band conditions of up to 877 g/in (see Table 1).

TABLE 1

| | | GRAMS | GRAMS | | | | |
|---|---|---|---|---|---|---|---|
| | WIDTH | PER LIN. | PER | THICKNESS | DENSITY | MACH. DIR | PERCENT |
| | MM | M | M² | (IN) | G/CC | TENSILE (G) | ELONGATION |
| Commercial Carded Band 70% Rayon, 30% | 50 | 9.70 | 194.00 | .094 | .081 | 572 | 15.65 |
| Staple Cotton | | | | | | | |
| Commercial A | 45 | 17.50 | 388.89 | .192 | .080 | 1600 | 20.47 |
| Commercial B | 47 | 8.90 | 189.36 | .103 | .072 | 877 | 42.15 |
| Commercial C | 53 | 10.00 | 188.68 | .136 | .055 | 320 | 16.35 |
| Commercial D Embossed | 53 | 10.10 | 204.60 | .089 | .095 | 235 | 18.35 |
| Commercial E Embossed | 50 | 11.20 | 212.35 | .096 | .087 | 568 | 9.25 |
| Commercial F Embossed | 52 | 12.80 | 257.30 | .116 | .089 | 525 | 13.72 |
| Example 1 Airlaid Web - 85% 1NR75 CC, | 52 | 10.40 | 200.00 | .081 | .097 | 561 | 28.72 |
| 15% Celbond BICO Fiber | | | | | | | |

Bands are sold in commerce by staple cotton bleachers and manufacturers of rayon fiber to tampon manufacturers where bands are available in composition ranging from 100% Cotton to 100% Rayon and in weight per unit area suitable for the various tampon production machines.

As can be seen from Table 1, the physical and performance characteristics of the airlaid tampon band of the present invention were comparable to those of the commercial and carded bands. Thus, it is now possible to make tampon bands with a high short fiber content that have strength and absorptive characteristics similar to those of more expensive bands made out of long staple cotton or rayon fibers.

Table II shows the results obtained by modifying the band of Example 1 to include varying proportions of short fiber to Bico fiber. Variables measured include basis weight (BW), density, tensile strength, tampon weight, fluid absorbance, and absorbency per tampon weight. In a preferred embodiment, the present invention provides a tampon including 10% Celbond Type 255, with 1-10% finished product moisture content. For tampon applications, absorptive capacity is determined by the Syngyna Absorbency Test as specified by the FDA, with capacities determined by product grade or size (e.g., Junior, Regular, Super or Super Plus). To pass the test a tampon of a given grade must fail after the maximum capacity for the grade is reached.

A series of prototype bands were produced on an M&J airlaid pilot machine. These tampon bands were converted into sewn pad style tampons. In a sewn pad style tampon, a rectangular pledget is formed from the tampon band. A nonwoven cover is wrapped around the pledget, leaving the short ends exposed. The withdrawal string is sewn down the center of the pledget in the long direction, holding the structure together. The tampon is then compressed in the lateral and longitudinal directions. Table II illustrates that a wide range of basis weights can be accommodated on an airlaid machine. Slightly higher levels of bicomponent fiber resulted in improved tensile strengths, even at low basis weights and densities. Higher basis weights require lower percentages of Bico fiber to maintain adequate tensile and band integrity. Fluid absorbency is primarily a function of density and is not impacted significantly by the percentage of Bico fiber within the range considered.

TABLE II

| | | | | | | Tampon | Tampon Fluid, |
|---|---|---|---|---|---|---|---|
| | | Basis | Dens- | Ten- | Tampon | Fluid | Total |
| | BICO | Weight | ity | sile | Weight | Absorbed | Absorbency |
| | % | (gsm) | (g/cc) | (g/in) | (g) | (g) | (g/g) |
| 1 | 9 | 204 | 0.094 | 463 | 2.6 | 11.5 | 4.4 |
| 2 | 9 | 204 | 0.060 | 281 | 2.6 | 12.2 | 4.7 |
| 3 | 7 | 210 | 0.098 | 217 | 2.8 | 11.5 | 4.2 |
| 4 | 7 | 199 | 0.042 | 108 | 2.6 | 12.2 | 4.5 |
| 5 | 9 | 814 | 0.062 | 1268 | 2.1 | 10.9 | 5.3 |
| 6 | 9 | 714 | 0.058 | 1726 | 2.4 | 11.4 | 4.8 |
| 7 | 7 | 691 | 0.071 | 1748 | 2.3 | 11.5 | 5.0 |
| 8 | 7 | 733 | 0.082 | 1115 | 2.5 | 11.3 | 4.6 |
| 9 | 5 | 669 | 0.075 | 616 | 2.3 | 11.0 | 4.7 |
| 10 | 5 | 590 | 0.052 | 544 | 2.1 | 11.1 | 5.3 |

### EXAMPLE 3

### Formation of a Tampon from Airlaid Tampon Band

The airlaid tampon bandstock of Example 1 (containing 85 percent cotton linter fibers and 15% Celbond Type 255 Bico thermoplastic fiber) was fed into a tampon making machine, Ruggli model no. TAR-CL-type 2. The band was drawn from a reel by rollers, pre-broken by localized perforation and stretching at lengths of 25-30 cm. A nonwoven cover sheet (of thermobonded polypropylene or bicomponent fiber approximately 8-9 g/m²) was drawn from a reel under tension. The non-woven web cover sheet was cut to length and welded to the tampon band by application of heat and pressure to the absorbent band. The length of the cover sheet was about one half the length of the band and was offset by about 3-4 cm behind the band to insure complete coverage of the absorbent plug after winding. The pre-broken band then moved to a knot tying unit where it was looped with the withdrawal string (carded, twisted 10/4 cotton string without finish). The band was then transported to the winder where it was broken by the first turn of the winder. The winder wound the band into a plug of about 25 mm diameter, having substantially concentric layers, covered by the nonwoven cover. The overlapping cover was then welded to the underlying cover sheet. The plug was then transported to the press. The press compressed the cylinder radially, and then compressed the cylinder longitudinally while maintaining the circumferential pressure to form a convex insertion end and a concave end to hold the withdrawal string. It was found that the press of the Ruggli tampon machine is capable of fusing the entire tampon together, preventing proper expansion when wet. Reduction of Bico fiber percentage, press temperature, and final compression are all ways of preventing complete fusion of the tampon structure. In this example, a 10% reduction in basis weight of the band compared to conventional material was used to decrease compression. Press heating was not used (ambient temperature pressing). Other combinations of reduced compression, reduced Bico fiber percentages, and reduced temperature were also found to work. The final dimensions of the tampon were 50.8 mm in length and 12.7 mm. in diameter. A tampon which comprises approximately 85 percent cotton linter fibers was produced which has physical and absorbancy characteristics comparable to tampons made from long staple fibers.

We claim:

1. A nonwoven, absorbent fibrous web, comprising:
a layer of airlaid substantially opened fibers, said fibers including more than 70% by weight short fibers; and
an interfiber binder.

2. The nonwoven, absorbent fibrous web of claim 1, wherein said short fibers are selected from the group consisting of cellulosic fibers, vegetable fibers, regenerated cellulose, synthetic long chain polymers, cotton linters, cotton noils, gin motes, long cotton and rayon fibers which have been reduced in length to 12 millimeters or less, and mixtures thereof.

3. The nonwoven, absorbent fibrous web of claim 1, wherein said short fibers are fibers of 12 millimeters or less in length.

4. The nonwoven, absorbent fibrous web of claim 1, wherein said interfiber binder is selected from the group consisting of latex emulsion, carboxymethylcellulose, cellulose acetate, poly (vinyl acetate), starch and thermoplastic binders.

5. The nonwoven, absorbent fibrous web of claim 1, wherein said interfiber binder is a thermoplastic binder selected from the group consisting of polyethylene, polypropylene, polyesters, polyamides, nylons, acrylics and mixtures thereof.

6. The nonwoven, absorbent fibrous web of claim 1, wherein said interfiber binder is a thermoplastic binder having a polyester core and an activated co- polyolefin sheath.

7. The nonwoven, absorbent fibrous web of claim 1, wherein said interfiber binder is a thermoplastic binder in a form selected from the group consisting of fibers, granules, pellets, powders, flakes, chips and mixtures thereof.

8. The nonwoven, absorbent fibrous web of claim 1, wherein said interfiber binder is water soluble.

9. The nonwoven, absorbent fibrous web of claim 1, wherein said layer of fibers includes between 70% and 99% by weight short fibers and a balance of long fibers.

10. The nonwoven, absorbent fibrous web of claim 9, wherein said layer of fibers includes between 80% and 95 % by weight short fibers and a balance of long fibers.

11. The nonwoven, absorbent fibrous web of claim 10, where said layer of fibers includes 93% by weight short fibers and a balance of long fibers.

12. The nonwoven absorbent fibrous web of claim 1, wherein said fibers are hydroentangled.

13. The nonwoven, absorbent fibrous web of claim 9, wherein said long fiber is selected from the group consisting of regenerated cellulose, staple cotton, rayon, and mixtures thereof.

14. The nonwoven, absorbent fibrous web of claim 5, further comprising a thermoplastic binder, selected from the group consisting of fibers, powders, granules, chips, flakes, and mixtures thereof.

15. The nonwoven, absorbent fibrous web of claim 1, exhibiting a machine direction tensile strength of between 100 and 1000 g/in., a density of less than 0.10 g/cc, and a percent elongation of less than 35.0%.

16. The nonwoven, absorbent fibrous web of claim 1, further comprising at least one material selected from the group consisting of super-absorbent polymer, perfumes, colorants, surfactants, antimicrobials, odor control agents and mixtures thereof.

17. A tampon, comprising absorbent material compressed into generally cylindrical form, said absorbent material including a nonwoven, absorbent fibrous web, including a layer of airlaid substantially opened fibers, said fibers including more than 70% by weight short fibers, and an interfiber binder.

18. The tampon of claim 17, wherein said short fibers are selected from the group consisting of cellulosic fibers, vegetable fibers, regenerated cellulose, synthetic long chain polymers, cotton linters, cotton noils, gin motes, long cotton and rayon fibers which have been reduced in length to 12 millimeters or less, and mixtures thereof.

19. The tampon of claim 17, wherein said fibers are hydroentangled.

20. The tampon of claim 17, wherein the interfiber binder is selected from the group consisting of latex emulsion, carboxymethylcellulose, cellulose acetate, polyvinyl acetate, starch and thermoplastic binders.

21. The tampon of claim 17, wherein the interfiber binder is a thermoplastic binder selected from the group consisting of polyethylene, polypropylene, polyesters, polyamides, nylons, acrylics, and mixtures thereof.

22. The tampon of claim 17, wherein the interfiber binder is a thermoplastic binder in a form selected from the group consisting of fibers, granules, pellets, powders, flakes, chips and mixtures thereof.

23. The tampon of claim 17, wherein the fibers include between 70% and 99% by weight short fibers and a balance of long fibers.

24. The tampon claim 23, wherein said fibers include between 80% and 95 % by weight short fibers and a balance of long fibers.

25. The tampon of claim 23, where said fibers include 93% by weight short fibers and a balance of long fibers.

26. The tampon of claim 17, wherein said long fibers are selected from the group consisting of regenerated cellulose, staple cotton, rayon and mixtures thereof.

27. The tampon of claim 17, wherein the web exhibits a machine direction tensile strength of between 100 and 1000 g/in., a density of less than 0.10 g/cc, and a percent elongation of less than 35.0%.

28. The tampon of claim 17, wherein the web further comprises at least one material selected from the group consisting of super-absorbent polymer, perfumes, colorants, surfactants, antimicrobials, odor control agents, and mixtures thereof.

29. A method for making a nonwoven, absorbent fibrous web, comprising:
providing a source of substantially opened fibers, said fibers including more than 70% by weight of short fibers;
introducing an interfiber binder to said source of fibers;
airlaying said source of fibers to form a layer of fibers; and
subjecting said binder to conditions sufficient to cause formation of interfiber bonds.

30. The method of claim 29, wherein said short fibers are selected from the group consisting of cellulosic fibers, vegetable fibers, regenerated cellulose, synthetic long chain polymers, cotton linters, cotton noils, gin motes, long cotton and rayon fibers which have been reduced in length to 12 millimeters or less, and mixtures thereof.

31. The method of claim 29, wherein said interfibers bands are formed by a method selected from the group consisting of thermal fusion bonding, adhesive bonding, solvent bonding, physical entanglement, hydroentanglement and combinations thereof.

32. The method of claim 29, further comprising introducing to said fibers a non-fibrous material selected from the group consisting of super-absorbent polymer granules, perfumes, colorants, surfactants, antimicrobials, odor control agents and mixtures thereof.

33. The method of claim 29, wherein interfiber binder is a thermoplastic fiber.

34. The method of claim 29, wherein the proportion by weight of cotton short fibers to thermoplastic fibers is between about 80/20 and 95/5.
